(19) United States
(12) Patent Application Publication
(10) Pub. No.: US 2006/0177409 A1
(43) Pub. Date: Aug. 10, 2006

(54) New cosmetic or dermatological use of ligans

(75) Inventors: Jean Pierre Arnaud, Verrieres Le Buisson (FR); Maria Anna Verbruggen, Renkum (NL); Rudolf Wilhelmus Maria Bakker, Bodegraven (NL)

Correspondence Address: STATTLER, JOHANSEN, AND ADELI LLP, 1875 CENTURY PARK EAST SUITE 1360, CENTURY CITY, CA, 90067, US

(73) Assignees: Lucas Meyer Cosmetics, Champlan (FR); Acatris, Londerzeel (BE)

(21) Appl. No.: 11/240,439

(22) Filed: Sep. 29, 2005

(30) Foreign Application Priority Data
Sep. 30, 2004 (FR) 04 10 370

(51) Int. Cl. A61K 36/55; A61K 8/97

(52) U.S. Cl. 424/74; 424/768

(57) ABSTRACT

The invention is related to the necessities of life, especially to cosmetology, pharmacology and, more specifically, to dermatology. This invention is the specific subject of a new use of lignans for making cosmetic, pharmaceutical and especially dermatological compounds, used to reduce sebum secretion through topical application. The invention also involves a cosmetic care process consisting of applying lignans, possibly in the form of cosmetic compounds, on the area of body and/or facial skin to reduce sebum secretion. Regarding this invention, the compounds are used in particular for the care and/or topic treatment of the body and/or face.

This application claims benefit to French Patent Application No. 04 10 370, filed Sep. 30, 2004, which is incorporated herein by reference.

The invention is related to the necessities of life, especially to cosmetology, pharmacology and, more specifically, to dermatology.

This invention is the specific subject of a new use of lignans for making cosmetic, pharmaceutical and especially dermatological compounds, used to reduce sebum secretion through topical application. The invention also involves a method of cosmetic care to reduce sebum secretion consisting of applying lignans, possibly in the form of cosmetic compounds in a container, on the affected area of body and/or facial skin.

Sebum plays an essential protective function in the body. Made up of lipids secreted by the sebaceous glands of the epidermis, sebum in fact makes up the hydrolipid film on the surface, providing the so-called barrier effect. When spread out on the skin's surface, it thus contributes to maintaining cutaneous hydration and protects the skin from outside chemical agents. A large number of internal and external factors control sebum secretion, which protects the skin according to the environment conditions. However, for a variety of reasons, excessive sebum may be secreted, which is known as “seborrhea”. Whether induced or inherent to a person's constitution, this seborrhea is not in itself a pathology and can be simply defined as excessive production of the sebaceous glands. However, this secretion, although initially beneficial to the skin, is aesthetically undesirable when excessive, and, moreover, often leads to uncomfortable, unsightly and even pathological skin problems.

Seborrhea has diverse origins. All external stressors that are hard on the hydrolipid film, such as pollution, sun, wind and heat, can trigger an exaggerated reaction by the skin, causing it to increase sebum production. The body's physiological state can also greatly influence this secretion. Thus, the inherent qualities of the skin, food imbalances, hormones, stress and sweating play major roles in seborrhea.

Excessive sebum production is unaesthetic, especially since the skin on the face appears shiny and thick, with dilated or blocked pores. When all over, it is the main characteristic of “oily” skin. This type of skin is also congested, often with areas of inflammation, especially pimples. When this skin is found with zones of dry skin, seborrhea is the sign of an imbalance characteristic of a combination complexion.

When the pilo-sebaceous pores are blocked, sebum can no longer be released. In this case, skin formations appear, such as blackheads, retentional or inflammatory acne, etc. Moreover, an inflammatory bacterial infection of the pilo-sebaceous follicle may result from this clogging: acne. Besides being infectious and retentional, excessive sebum secretion is an established characteristic of acne, whether induced or inherent to a person's constitution. The proliferation of germs in a blackhead can lead to inflammation, irritation and even the formation of new sebaceous micro-cysts. In the pilous follicle, excessive sebum production gives hair that unsightly greasy look, and, in the long term, may stifle the pilous bulb and cause the premature loss of hair.

Regularly cleaning the skin to eliminate excessive sebum and preventing the obstruction of the sebaceous glands is not a satisfactory or sustainable solution. In fact, it can often make the epidermis more fragile and result in seborrhea reacting, for instance, to the use of astringents.

Keratolytic agents, such as benzoyl peroxide, are commonly used to clean out blocked pores, especially in the treatment of acne. However, these aggressive treatments, in addition to their many contra-indications, are not the best solution for treating reactive skin and are often poorly tolerated by sensitive skin. As well, vitamin A derivatives, such as oral isotretinoin or topical tretinoin effectively reduce sebum production, but are cumbersome treatments and difficult to keep up due to the resulting skin dryness and various contra-indications. These long-term treatments take a long time for results to become visible.

Hormonal regulation is often recommended in treating acne. The excessive secretion of sebum that accompanies juvenile acne in particular is the symptom of excessive secretions in response to hormonal activity. Administering estrogens and/or anti-androgens, such as cyproterone acetate, is used in oral contraceptive treatments. Along with antiseptic or antibiotic agents, they are part of a classic therapeutic arsenal for treating this type of acne. However, excessive sebum secretion should be treatable without having to resort to hormonal, antiseptic or antibiotic treatments that have side effects, are taken orally or for which the long-term use is not desirable. The efficacy of topical hormonal treatments is often questionable.

Many studies have been conducted in the area of cosmetics to identify compounds that could significantly reduce excessive sebum secretions. One problem lies with the nature and tolerance of the care. Indeed, seborrheic skin requires specific care due to its comedogenic nature. Oily skin especially is very easily stressed and irritated and requires care specifically adapted to its reactive and sensitive nature. Sebo-regulators must therefore be easy to formulate and to be compatible with many different types of care, in particular with light emulsions.

Research has focused on an enzyme that is highly active in the regulation of sebum secretion: 5α-reductase. This enzyme specifically converts testosterone into dihydrotestosterone, an androgen needed for sebaceous gland function. In particular, it is known that increased activity of this enzyme may be a reason for excessive sebum secretion and follicular hyperkeratinization often found in oily skin.

There are two different iso-forms of 5α-reductase, which only have 50% homology in their amino acid sequence. The Type 1 iso-enzyme is primarily found in the liver, skin and especially in the sebaceous glands. Type 2 iso-enzyme plays a role in sex differentiation and is predominantly found in the prostate and skin around the areas of sexual differentiation.

A large number of compounds have thus been identified as being 5α-reductase inhibiters. Linolenic and linoleic acids (Avon Products WO00/13661) and fatty-acid esters (Pharmascience Lab, WO0152837 A3) have been described in terms of their inhibitory action against 5α-reductase. Conjugated linoleic acid has also been described in Patent Application WO01/08651 (Unilever) as an agent to regulate sebum secretion and used in combination with a wide variety of phenolic agents, such as xanthans, isoflavones, lignans and lignin. Steroid derivatives such as 17-beta-cyclopropyl(amino/oxy)4-aza steroids (EP0880540B1, Hoechst Marion Roussel Inc.), 0-fluoro-17(20)-vinyl steroids (WO0200681, Burkhart, et al.), 4-aza 17-β 5α androstan-3one.

Steroids (U.S. Pat. No. 4,732,897, Erab, et al.) were described for their inhibitory activity of 5α-reductase. Steroids and their derivatives are however tricky to use and their rapid metabolism does not provide a sustainable inhibitory effect.

Finasteride has long been the benchmark in terms of a competitive steroid compound used in the treatment of prostate cancer (J. Med. Chem 36, 4313-(1993)). However, it has not proven to be very active on Type 1 iso-enzyme. Patent EP0880520B1 (Applied Research Systems) describes benzo-quinolizine derivatives that can specifically inhibit Type 1 iso-enzyme.

Plant extracts, such as extracts from the palm tree, Serenoa repens, Cavalia gladiata, Biota orientalis and Coptis chinensis also showed inhibitory activity on 5α-reductase. These extracts, in addition to their instability, are not always tolerated by the skin, mainly because of their solvent content.

The Applicant has recently discovered, quite unexpectedly and by surprise, that lignans, and more specifically secoisolariciresinol diglycoside (SDG), are cosmetic or dermatological agents that can significantly reduce sebum secretion. Without limiting the invention to this hypothesis regarding this mode of action, lignans seem capable of inhibit 5α-reductase, in particular the Type 1 iso-enzyme through topical application. These natural compounds have the advantage of being well tolerated by the skin and easy to formulate, especially as a light emulsion. Lignans are a perfectly suitable solution to the needs of the skin without the aforementioned disadvantages.

Lignans have been largely used in the oral treatment of a number of diseases, in particular diabetes and hypercholesterolemia (U.S. Pat. No. 5,846,944, University of Saskatchewan), cardiovascular diseases (CA2311606, University of Saskatchewan) and for their anti-cancer or phyto-estrogen properties (WO821946, Internutria). Lignans in combination with isoflavones have, for example, been described as useful food supplements, especially in the treatment of dementia, migraines, prostate cancer and premenstrual syndrome (EP0906761B1, Archer Daniel Midland). In combination with carbohydrates, they have been described as a source of phyto-estrogen for the oral treatment of gynecological problems related to menopause. Their properties are apparently mostly the result of their metabolization by intestinal flora into enterolactone and enterodiol whose structure is related to certain estrogenic compounds.

Moreover, lignans have already been used in topical applications, especially due to their antioxidant properties. These compounds, in particular hydroxy-matairesinol and matairesinol, were thus used in the treatment of a number of diseases mediated through free radicals (WO2003/45376, Hormos Nutraceutical Ltd.). Patent Application WO2004/000304 A1 (Hormos Nutracentical Ltd.) describes the use of lignans, in particular secoisolariciresinol algycon, potentially in the form of a lipid ester, to be used in cosmetic or pharmaceutical compounds having antioxidant activity. Patent Application WO2004/010965 (L'Oréal) describes the use of aglycon lignans, obtained through acid hydrolysis, as an anti-aging agent. These aglycon lignans were described for their cosmetic properties in the treatment of skin dryness and redness, in improving skin tone and in softening skin (WO2004012697 L'Oréal).

The main source of lignans is flax or Linum usitatissiumum, which is also used in cosmetics. However, the sebo-regulator properties of flex, especially the lignans it contains, had up to now never been described. Having conducted active studies on lignans, the Applicant has recently noticed that these natural compounds are excellent anti-seborrheic cosmetic and dermatological agents.

Therefore, the purpose of this invention is to use lignans to produce a cosmetic or dermatological compound that, through topical and preferably cutaneous application, will be used to reduce sebum secretion.

Lignans are a group of low-molecular weight bicyclical chemical compounds containing a 2-3 dibenzylbutane structure (Merck Index). More specifically, in the context of the invention, the term “lignan” is based on IUPAC nomenclature in its strictest sense, i.e., compounds and compound derivatives, in particular in the form of glycosylate, whose structure is the result of binding together of the β carbons of the lateral chains of two units of propylbenzene (8-8′ bond), with the exception of common cytotoxic compounds, such as podophyllotoxin (Dictionnaire des Sciences Pharmaceutiques et Biologiques, Académie Nationale de Pharmacie, Ed. Louis Pariente, 1997, p. 387). In this case, they are compounds having the following basic structure (I): 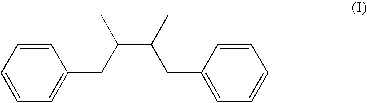
and that may contain substitution groupings, especially of the following types: oxide, hydroxyl, alkyl, alkoxy, and, in particular, methoxy. Among the derivatives, there are primarily mono and diglycosyl derivatives. Plant lignans can be metabolized through enzymatic reactions into so-called mammal lignans, such as enterolactone and enterodiol, also included in this invention. Among the lignans, secoisolariciresinol diglucoside or SDG (CAS 148244-82-0), matairesinol, hydroxymatairesinol or HMR, lariciresinol and nortrachelogenin are compounds that are especially effective in reducing sebum cutaneous secretions. Neolignans, lignan oligomeres, C17 norlignans and lignoids are therefore not included in this definition.

Lignans may be obtained through chemical synthesis (Synthesis of First Lignans Found in Man and Animals: G. Cooley, et al., Tetrahedron Letters 22, 349 (1981)). Lignans may also be used in a form of plant extract in containers. Based on one of the preferred processes in the invention, lignans will be in the form of a hydro-alcoholic extract of flax seeds.

Based on the invention, lignans may be obtained by methods classically described in previous related work. For example, we can refer to the process in Patent Application WO03/75686 (Suntory Limited). According to the process described in U.S. Pat. No. 5,705,618 (Westcott and Muir), extraction using the aliphatic alcohol of de-fatted flax seeds very easily allows to obtain a concentrate of 95% SDG.

Based on the invention's preferred method, lignans will be obtained by maceration of the flax seed husk in a heated hydro-alcoholic solvent, and then by separation of the solid residue from the lipophilic impurities. Flax seed husks can be isolated in a variety of ways, particularly mechanical, and, preferably, by grinding the de-fatted seeds and then retrieving the fraction rich in husks through its density (density fraction 20-50 kg/hl) or using granulometry. This lignan extraction process then involves a step in which the flax seed husk is macerated in a solvent, ideally made of a combination of water, methanol, ethanol, propanols, butanols and/or their mixes. Based on the preferred method, the solvent is a 50-50 hydro-alcohol mix.

This maceration is carried out between 30 minutes and two hours at a temperature of at least 30° C., preferably between 40° C. and 60° C. Under these field conditions, an extract:husk extraction ratio of between 1:20 and 5:20 can be obtained. The solid residue is then removed by filtration. A separation step then eliminates lipophilic impurities. This separation method is ideally carried out in accordance with the procedure described in Patent EPO 730 830 B1 (Emil Flachsmann AG). The procedure described in this patent involves mixing the extract containing lipophilic impurities with a lipophilic phase for approximately one hour. The lipophilic impurities are then removed with the lipophilic phase through the separation of phases, preferably on a synthetic membrane. The extract containing lignans is in the hydrophilic phase and can be concentrated and/or purified using traditional methods, in particular those described in U.S. Pat. No. 5,705,618 (Westcott and Muir). Preferably, the extract is then concentrated using distillation and then dried at ultra-high temperature.

Under these conditions, a concentrate preferably of 20% lignans, especially of SDG, may be obtained. The extract also contains sugars, primarily glucose. Its precise composition depends on the selected operating parameters. The composition of a particularly interesting extract according to the invention and subject to tests is shown in detail in Example 1.

The studies, presented in Example 2, have shown the sebo-regulator properties of lignans, especially as an extract obtained from flax seed husks under predefined conditions. They show that sebum production is significantly reduced after 14 days of treatment and is even further reduced with longer treatment. It is thus reduced by 20% versus a non-treated area of skin after 28 days of treatment. The people in the tested sample, all of whom initially had oily skin, had normal complexions after 28 days of treatment. The number and size of pimples are reduced after only 14 days of treatment. Skin lesions are also diminished. Moreover, lignans are not comedogenic. Lastly, according to the invention, lignans are especially interesting as cosmetic or dermatological agents for reducing sebum secretion, in particular when this secretion is excessive. Based on the stated hypothesis, and without the use based of the invention being limited to this method of action, the inhibition of sebum secretion could be achieved by inhibition of the activity of Type 1 5α-reductase.

Based on the invention, lignans are used preferably in a concentration of between 0.002% and 5% of the total compound weight. They are ideally used in a concentration of between 0.02% and 1% of total compound weight.

In the context of the invention, “topical application” refers to application on the scalp and skin, especially the skin of the body and face.

Cosmetic and dermatological compounds containing lignans are produced in the usual manner. Lignans, when they are used as a dry extract are first dissolved in warm water before being mixed. The compound's pH will be higher than 4.5. They can contain other cosmetically or dermatologically acceptable ingredients. Based on a preferred method for producing cosmetic or dermatological compounds, lignans will be used in combination with restructuring agents, such as phospholipids, other classic sebo-regulators and/or sebum-absorbing agents. Restructuring agents could include phospholipids, especially the Heliogel® product (Lucas Meyer Cosmetics), made up of sodium acrylate copolymer, hydrogenated polyisobutane, phospholipids, polyglyceryl 10-stearate, and sunflower seed oil (Helianthus annuus), as described in Patent WO2004030605 (Lucas Meyer Cosmetics). The addition of classical sebo-regulator agents, such as phytosterols, and, specifically isoflavones, reinforces the sebo-regulator effect. The compounds, as per the invention, will ideally contain sebum-absorbing agents, and preferably the agent marketed under the name Matipure® (Lucas Meyer Cosmetics) made up of a mix of magnesium/aluminium silicate, cellulose hydroxyethyl, black cumin (Nigella sativa), squash seed oil (Cucurbita pepo), and phospholipids.

Compounds of these types can also contain cosmetically or pharmaceutically acceptable, common or useful excipients or diluents, in particular as diluting agents, dispersant agents, gelling agents, solid emollients, gums, resins, solvents, fillers such as modified and polymerized starches, titanium dioxide or metallic stearate, conserving agents, essential oils, pearl agents, colouring, odour absorbers, pH-regulator agents or neutralizing agents, thickening agents, absorption promotion agents, aromatic or perfume agents, sun protection agents, especially micronized metallic oxide particles, metallic silicate agents or organic compounds (tetramethyl butyl phenol bis-benzotriazolyle, ethylhexyl methoxycinnamate or butyl methoxydibenzoylmethane), mineral pigments such as iron oxides, oily agents such as plant-based grease or oil, synthesis oils (perhydrosqualen), silicon oils (cyclomethicone), fluorinated oils (perfluorinated polyethers, perfluorodekalin), esters, fatty alcohols (cetyl alcohol), waxes (carnauba wax, Montana wax, ozocerite, Percilla wax), modified clays, Bentones, fatty acid metallic salts, hydrophobic silica, polyethylenes, mica or other substances used in cosmetics.

The choice and/or quantity of complementary ingredients in the compound will also be determined based on tolerance and specific needs of the skin on which the compound will be applied as well based on the properties and desired consistency for the invention's compound. Different reagents are used in the same proportions as used generally in cosmetics, for example, 0.01 to 20% of total compound weight.

The compounds used with this invention are in the appropriate form for topical application, and preferably cutaneous for men and/or animal. They are in the form of gel, cream, lotion, emulsion or dispersion, especially oil in water or water in oil, multiple emulsions, ointment, milk, foam: spray, patch or impregnated fabric. They will be preferably used in a fine oil-in-water emulsion.

The purpose of the invention is using lignan cosmetics, preferably in the form of the cosmetic compound predefined in the container to reduce the sebum secretion and/or the care, prevention and/or cosmetic treatment of excessive secretion of sebum and its unsightly and/or uncomfortable symptoms, especially for reducing the shiny and/or blocked appearance of the skin, preventing and/or reducing the size or number of cutaneous lesions related to seborrhea, the formation of retentional or inflammatory acne, blackheads and/or diminishing imperfections. The purpose of the invention is also for use in lignan cosmetics, preferably as a cosmetic compound predefined in a container, for the care, prevention and/or treatment of oily hair and/or hair loss.

In the context of the invention, the compounds are especially suited for producing anti-seborrheic cosmetic products for the care and/or cosmetic treatment of seborrheic skin, especially skin that is oily, that tends to oily or is combination.

The purpose of the invention is also for the dermatological use of lignans, preferably as a predefined dermatological compound to produce a topical medication to prevent and/or treat pathologies and/or cutaneous disorders related to excessive sebum secretion, in particular, for the prevention and/or treatment of the seborrheic component of acne, and in particular blackhead formation.

Moreover, the invention also involves a cosmetic care procedure consisting of applying lignans, preferably as a predefined cosmetic compound, on the area of skin concerned, to reduce sebum secretion and/or the unsightly signs of excessive secretion. This cosmetic treatment method is therefore particularly suited to the treatment of oily and/or combination and/or acne seborrheic.

The compounds for this invention are non-toxic and are well-tolerated in the application area. They are also non-allergenic.

The following examples are presented to illustrate the invention and must in no ease be considered to limit the scope of the invention. Unless otherwise indicated, concentrations are given as a percentage of total compound weight.

### EXAMPLE OF PRODUCING A USEFUL EXTRACT BASED ON THE INVENTION

The extract tested hereafter, which contain lignans, was obtained based on the following protocol:

Flax seeds are selected.

Seed husks are separated.

Husks are left to macerate in a water-ethanol solvent in a 50-50 ratio at approximately 40° C. for two hours.

Solid residue is eliminated through filtration.

Based on the separation procedure described in Patent EP073083B1, the extract is mixed during the lipidic phase at a temperature of approximately 70° C. during approximately one hour. The separation of phases is done using a synthetic separation membrane.

The extract obtained during the hydrophilic phase containing the lignans is concentrated through distillation, and undergoes ultra-high temperature treatment.

Flax extract thus obtained is dried.

The flax extract thus obtained is in the form of a dark orange powder, titrated at 20% of lignans, primarily of SDGs, and is a compound whose concentrations of other components vary within the following percentage ranges.

Extract weight compound:

| | |
|---|---|
| SDG | 15-30% |
| Water | 0-10% |
| Protein | 0-15% |
| Lipids | 0-10% |
| Fibres | 0-5% |
| Sugars | 2-20% |
| | (mainly glucose) |
| Maltodextrin | 0-35% |

This extract may be directly formulated, after pre-dispersion in warm water in compounds with a pH lower than 4.5.

The oil emulsion in the water tested in Study II was produced as follows:

Composition in Percent:

| Phase | Ingredient | Suppliers | in % |
|---|---|---|---|
| A | Demineralized water | water | 75.4% |
| | butylene glycol | butylene glycol | 3.00 |
| | glycerin | glycerin | 2.00 |
| B | Heliogel ® | sodium acrylate copolymer + | 2.00 |
| | | hydrogenated polyisobutane + | |
| | | phospholipids +polyglyceryl | |
| | | 10-stearate | |
| | | Helianthus annuus seed oil (Lucas | |
| | | Meyer Cosmetics) | |
| C | Dermofeel ® | butyleneglycol dicarprylate/ | 4.00 |
| | | dicapate | |
| | DC 345 | cyclopentasiloxane | 2.00 |
| | Vitamin E acetate | tocopheryl acetate | 0.10 |
| | hydrogenated | hydrogenated palm oil | 2.00 |
| | palm oil | | |
| | phenonip | phenoxyethanol, esters, paraben | 0.50 |
| D | Demineralized water | water | 10.00 |
| | flax seed extract as | | 1.00 |
| | per invention | | |

Operating Procedure

Phases A and B are heated separately at 70-75° C., stirred, until they are completely homogenous.

Phase B is slowly introduced into Phase A, gently stirring.

Phase C is heated at 70-75° C. is added slowly to the mix, stirring vigorously.

Phase D is heated separately until 60-65° C., stirring vigorously and constantly until completely mixed and homogenous, and then added to the mixture while gently stirring.

Everything is allowed to cool down while gently stirring.

Study II In Vivo Study of Lignan Activity on Skin Sebum Regulation

Principle:

The objective of the study is to assess non-comedogenic activity, cutaneous tolerance as well as sebo-regulator activity of a cosmetic product for oily skin applied under normal conditions of use for 28 days by healthy adult volunteers.

Procedure:

A population of 20 women with oily skin were treated for 28 days. A water-in-oil emulsion containing 1% of flax extract previously described in Study I, i.e., 0.2% of lignans, is applied twice a day on the entire face.

Cutaneous examinations, notably for cutaneous tolerance and blackhead counts were carried out by the investigating dermatologist at the beginning (D0) and at the end (D28) of the study for each volunteer. During these examinations, the doctor noted the appearance of the skin and counted the number of acne lesions (blackheads, papules, pustules, nodules, etc.) before and after treatment. To evaluate tolerance, the investigator used several criteria: erythema, oedema, dryness, desquamation, etc. Subjective tolerance of the product was also assessed by the volunteers and discussed again during the last visit (D28) with the investigating physician.

The sebum rate was measured using a procedure based on the Sebumeter principle, which involves directly measuring the sebaceous secretion using a photometric method regardless of the hydration measurement.

The Sebumeter principle is based on the application of an unpolished film on the surface to be measured. The head of the cassette, on which the film is set, is inserted into a measuring tube and a cell analyzes its transparency. The light transmitted represents the level of sebum contained in the area measured. The sebum level measured this way is expressed in μg of sebum/cm² of skin. Many bibliographic references report that, using this technique, people with combination-to-oily skin have a sebum level between 100 and 220 μg/cm², and the oily to very oily skins have a sebum level higher than 220 μg/cm². Based on this information, volunteers were recruited.

Sebum levels were also measured using Sebutape® patches. To do this, the skin was first cleaned 30 minutes before the beginning of the test using a towelette containing alcohol. As well, the volunteers did not apply any cosmetic product (make-up, cream, etc.) on their face before each measurement session. The Sebutape® patches were put on using a pad, which provided the same pressure for all volunteers. The patches were kept in contact with the skin for 15 seconds. The Sebutape® patches were made of a proteic film, which, upon contact with the sebum, deteriorates. Spots, representing the skin sebum rate, appear on the surface of the patches. Each spot corresponds to a pore and a sebaceous gland, which allows to see the size and number of active sebaceous glands.

Results Obtained for Sebum Production

The results were expressed in percentage in relation to the untreated area.

| | Time | | | |
|---|---|---|---|---|
| | 7 days | 14 days | 21 days | 28 days |
| Average variation in | −8 | −14 | −18 | −20 |
| sebum production (in %) | | | | |
| Significance at 95% | NS | S | S | S |
| (p < 0.05) | | | | |

S: The results are significantly different from that obtained in the control group, based on the p value.
NS: The results are not significantly different from that obtained in the control group, based on the p value.

The application of the compound containing lignans leads to decreased sebum production. This decrease is significant at 95% of the time starting the 14th day and thereafter.

After one month of treatment, sebum production is reduced by 20% versus the untreated area of skin.

At the beginning of the test, 100% of the test population had a sebum levels of between 100 and 200 μg/cm², and therefore the skin was considered to be oily. After 28 days of treatment, 50% of them had “normal” skin with a sebum levels below 100 μg/cm².

Results Obtained for Pimples:

The results were expressed in percentage in relation to the untreated area.

| | Time | | | | |
|---|---|---|---|---|---|
| | Initial T | 7 days | 14 days | 21 days | 28 days |
| Size of pimples | 0.48 | 0.44 | 0.33 | 0.23 | 0.22 |
| (in mm) | | | | | |
| Significance at 95% | NS | NS | S | S | S |
| (p < 0.05) | | | | | |

S: The results are significantly different from that obtained in the control group, based on the p value.
NS: The results are not significantly different from that obtained in the control group, based on the p value.

Starting on the 14th day, 70% of the population treated experienced a decrease in the number of pimples. After 21 days of treatment, 80% of the volunteers saw a reduction in the number of pimples. This reduction is 25% versus the untreated area of skin. In 28 days, 90% of the population treated saw a decrease in the number of pimples. This number decreased by 34% and the average size of the pimples went from 0.48 mm to 0.22 mm, and therefore shrank by 54.2%.

Evaluation of the Comedogenic Effect

At 28 days, 79% of the test population showed a 34% reduction in the number of skin lesions. After one month of treatment, the product can be considered to be non-comedogenic.

Conclusion:

Flax extract, and especially the lignans it contains, have sebo-regulator activity that can be significantly observed after 14 days of treatment. This activity is reinforced with the length of treatment and is correlated to the reduction in number and size of pimples as well as in a significant reduction in skin lesions. This effect is also free of any comedogenic action.

II Examples of Compounds

Light Gel-Cream Sebo-Regulator:

Composition in Percent:

| Phase | Ingredient | Suppliers | in % |
|---|---|---|---|
| A | demineralized water | water | qs 100% |
| | Dermofeel PA 3 | sodium phytate and water | 0.05 |
| | Heliogel ® | sodium acrylate copolymer + hydrogenated | 3.00 |
| | | polyisobutane + phospholipids + polyglyceryl | |
| | | 10-stearate | |
| | | Helianthus annuus seed oil (Lucas Meyer Cosmetics) | |
| B | Dermosoft LP | caprylyl glycol, glyceryl caprylate, | 0.50 |
| | | glycerin, phenyl propanol and water | |
| | | (Lucas Meyer Cosmetics) | |
| C | Dub Inin | Isononyl isonanoate | 2.00 |
| | DC 345 | cyclopentasiloxane | 3.00 |
| | Dermofeel ® BGC | butyleneglycol dicarprylate | 3.00 |
| | | dicaprate | |
| | Vitamin E acetate | tocopheryl acetate | 0.50 |
| | hydrogenated palm oil | hydrogenated palm oil | 2.00 |
| | phenonip | phenoxyethanol, esters, paraben | 0.50 |
| D | colouring | CI 42090 | qs colour |
| | colouring | C1 19140 | qs colour |
| E | Demineralized water | water | 15.00 |
| | flax seed extract as per invention | | 0.50 |
| | Matipure | magnesium aluminium silicate, | 2.00 |
| | | hydroxyethylcellulose, Nigella sativa seed | |
| | | oil, Cucurbita pepo seed oil, phospholipids | |
| | | (Lucus Meyer Cosmetics) | |
| F | fragrance | C 3810 | 0.40 |

Operating Procedure

1. Phases A and B are heated separately at 70-75° C., stirred, until they are completely homogenous.

2. Phase B is slowly introduced into Phase A, gently stirring.

3. Phase C is heated at 70-75° C. is added slowly to the mix, stirring vigorously.

4. Phase D is added.

5. Phase D is heated separately until 60-65° C., stirring vigorously and constantly until completely mixed and homogenous, and then added to the mixture while gently stirring.

6. Everything is allowed to cool down while gently stirring.

7. When the temperature drops lower than 35° C., Phase F is added.

8. pH is adjusted if necessary.

Specific Care for Oily Skin, Tone Refiner

Composition in Percent:

| Phase | Ingredient | Suppliers | in % |
|---|---|---|---|
| A | demineralized water | water | qs 100% |
| | glycerin | glycerin | 3.00 |
| | xanthan gum | xanthan gum | 0.50 |
| | Dermosoft LP | caprylyl glycol, glyceryl caprylate, | 1.50 |
| | | glycerin, phenyl propanol and water | |
| | | (Lucas Meyer Cosmetics) | |
| B | Amisol Soft | behenyl alcohol, glyceryl stearate, lecithin, | 4.00 |
| | | glycine soya sterol | |
| | | (Lucas Meyer Cosmetics) | |
| | Cetiol SB 45 | butyrospermum parkii | 5.00 |
| | Dermofeel ® BGC | butyleneglycol dicaprylate dicaprate | 5.00 |
| | Nipanox BHT | BHT | 0.10 |
| | Dub Inin | Isononyl isonanoate | 1.00 |
| | DC 345 | cyclopentasiloxane | 3.00 |
| C | Flax extract | | 0.50 |
| | demineralized water | water | 10.00 |
| D | Covasop white colouring | CI77891, propylene glycol | 8.00 |
| | Covasop yellow colouring | CI77492, propylene glycol | 1.76 |
| | Covasop red colouring | CI77491, propylene glycol | 0.50 |
| | Covasop black colouring | CI77499, propylene glycol | 0.15 |
| E | SJ touch | PMMA | 4.00 |
| F | fragrance | C 304323 | 0.30 |

Operating Procedure:

1. Phases A and B are heated separately at 70-75° C., stirred, until they are completely homogenous.

2. Phase B is slowly introduced into Phase A, gently stirring for 20 minutes (phospholipids hydration time).

3. Everything is homogenized while stirring vigorously (stator rotor) at 3,000 RPM for three minutes.

4. Phase C, which was previously heated at 60° C., while gently stirring, is added to the mix while vigorously stirring for two minutes.

5. When the temperature drops lower than 35° C., Phase D, E and F are added.

1. Use of lignans for producing a topical cosmetic or dermatological compound to reduce sebum secretion.

2. Use of lignans, according to claim 1, in which lignans are in the form of a hydroalcoholic flax seed extract.

3. Use of lignans, according to claim 1, in which this extract is obtained through maceration of flax seed husks in a heated hydroalcoholic solvent, then through the separation of solid residues and lipophilic impurities.

4. Use of lignans, according to claim 1, in which this extract is concentrated at 20% in lignans.

5. Use of lignans, according to claim 1, in which the lignans are secoisolariciresinol diglucoside.

6. Use of lignans, according to claim 1, at a concentration between 0.002% and 5% in total compound weight.

7. Use of lignans, according to claim 1, at a concentration between 0.02% and 1% in total compound weight.

8. Use of lignans, according to claim 1, to produce a cutaneous cosmetic or dermatological compound to reduce the sebum secretion via the skin.

9. Use of lignans, according to claim 1, in combination with restructuring agents, such as phospholipids, other classic sebo-regulator agents and/or sebum absorbing agents.

10. Cosmetic use of lignans, according to claim 1, to inhibit Type 1 5α-reductase.

11. Cosmetic use of lignans, according to claim 10, to reduce the secretion of sebum and/or for the care, prevention and/or cosmetic treatment of excessive sebum secretion and its unsightly and/or uncomfortable symptoms.

12. Cosmetic use of lignans, according to claim 10, for the care, prevention and/or treatment of oily hair and/or hair loss.

13. Cosmetic use of lignans, according to claim 10, to produce anti-seborrheic products for the care and/or treatment of seborrheic skin, oily skin, skin with oily tendencies or combination skin.

14. Use of lignans, according to claim 1, to produce a topical drug for the prevention and/or treatment of pathologies and/or skin disorders related to excessive sebum secretion, in particular for the prevention and/or treatment of the seborrheic component of acne.

15. Method of cosmetic care using lignans, consisting of applying lignans on the area of skin concerned to reduce sebum secretion and/or unsightly symptoms of excessive sebum secretion.

16. Method to reduce sebum secretion by using lignans in a topical cosmetic or dermatological composition.

17. Lignans from flax to reduce sebum production.

18. Compositions for topical cosmetic or dermatological use containing lignans at a concentration between 0.002% and 5% in total compound weight.
